(19) United States
(12) Patent Application Publication
(10) Pub. No.: US 2015/0050727 A1
(43) Pub. Date: Feb. 19, 2015

(54) PURIFICATION

(71) Applicant: Imperial Innovations Limited, London (GB)

(72) Inventors: Joshua Benno Edel, London (GB); Vladimir Alexander Turek, London (GB); Anthony Edward George Cass, London (GB)

(21) Appl. No.: 14/459,015

(22) Filed: Aug. 13, 2014

(30) Foreign Application Priority Data
Aug. 14, 2013 (GB) 1314581.8

(51) Int. Cl. C12Q 1/68

(52) U.S. Cl. CPC C12Q 1/6806
USPC 435/320.1; 536/25.4

(57) ABSTRACT

The invention provides methods of purifying one or more biological analytes from a sample. The method comprises incubating the sample with nanoparticles and centrifuging a biphasic system. The biological analytes can be one or more of an enzyme, a protein, a nucleic acid, an organelle, a cell, a bacteria, a virus, or any other biological material which requires purification.

### RELATED APPLICATIONS

The present application claims priority under 35 U.S.C. §119(a) to Great Britain application No. 1314581.8, filed Aug. 14, 2013, which is incorporated herein by reference.

The present application relates to an improved method of purifying biological analytes.

The isolation and purification of biological material is well established in the art. The provision of biological material in an isolated and purified state is important for the preparation for example of enzyme assays to test new drugs or to detect the presence of bacterial or viruses in a sample.

Many techniques have been developed in the art for the purification of biological material, including chromatography, such as size exclusion chromatography or affinity chromatography.

One method of purification involves the use of magnetic beads which are used to purify biomolecules and cells. The magnetic particles are surface modified to provide an affinity group which binds to a target biomolecule or cell. A magnet is then used to immobilise the magnetic particles and the trapped analytes, allowing the removal of the unbound material and the resulting purification of the target biomolecule or cell.

There are however some issues with this method. In particular magnetic beads tend to be expensive and require multiple repeat steps to achieve a good sample purity. The use of magnetic beads can also lead to non-homogenous separations. In particular, beads which are close to the magnets will experience strong magnetic fields and forces. This can result in irreversible aggregation problems if the magnetic forces are too high and/or the exposure time in the magnetic field is too long. Conversely any beads which are further away from the magnets will experience weaker magnetic forces and magnetic fields and will require longer times to reach the retention positions which can result in a loss of magnetic beads.

There is provided by the present invention an improved method of isolating and purifying a biological analyte.

The first aspect of the present invention provides a method of purifying a biological analyte from a sample comprising;
incubating a sample comprising a biological analyte with nanoparticles
adding an organic phase or an aqueous phase as necessary to produce a biphasic system
centrifuging the mixture until a sedimented aqueous droplet comprising the nanoparticle bound biological analyte is formed; and
separating the resulting sedimented aqueous droplet
   wherein the organic phase is immiscible with the aqueous phase and has a density greater than 1.0 g/cm³.

The method of the first aspect results in the formation of a liquid pellet (the sedimented aqueous droplet) which does not disperse into the bulk aqueous or organic liquid phases. Separation of the sedimented aqueous droplet can be carried out in any manner known in the art for example by decanting the supernatant or by directly removing the droplet (for example with a pipette). The sedimented aqueous droplet can be diluted, if required to provide a purified solution of the biologically analyte bound to the nanoparticle or can be combined with other sedimented aqueous droplets to increase the content of the biological analyte even further, while keeping the concentration of impurities approximately consistent.

It will be appreciated that the method of the present invention provides a one step method of both isolating and purifying a biological analyte from a sample. The selective binding of the biological analyte by the nanoparticles and the formation of the sedimented aqueous droplet containing the nanoparticle and the bound analyte allows the biological analyte to be removed from the sample (i.e. to be isolated) in a purified form (as the sedimented aqueous droplet contains the nanoparticles and bound biological analyte). Thus any reference in the specification to purifying the biological analyte also includes the isolation of the analyte.

For the purposes of this invention, the biological analyte is not limiting and can be one or more of an enzyme, a protein, a nucleic acid (including DNA including cDNA, RNA particular mRNA or a plasmid), an organelle, a cell, a bacteria or a virus or any other biological material which requires purification.

The method of the first aspect of the invention can be carried out with any nanoparticles which have a higher density than the phase they are dissolved in. Examples of such nanoparticles include metal oxide types, gold, silver and silica colloids and quantum dots

The particles may be further functionalized by ligands such as specificity ligands in order improve the efficiency of the method. Examples of functionalities include, but are not limited to antibodies, aptamers, proteins, modified enzymatic substrates and small chemicals with a high affinity to precious (bio)materials. Methods of functionalising nanoparticles are known in the art and include these described in Javier et al, Bioconjugate Chem. (2008), 19, 1309-1312, Lee et al, JACS, (2012), 134, 1576-15772 and Chang et al, Scientific Reports (2013), 3 (1863) 1-7.

The biological analyte can bind to the nanoparticles either directly or via a ligand by electrostatic or covalent binding. Removal of the biological analyte from the nanoparticle or ligand can be carried out using methods known in the art.

As discussed above, the method involves the formation of a mixture having an organic and an aqueous phase. For the purposes of this invention, the term “aqueous” is intended to cover any liquid phase which is immiscible with the organic phase and has a different, preferably lower density than the organic phase. The aqueous phase can be water, or a salt solution, such as a NaCl solution or a buffer solution, such as a phosphate buffer solution or a TBE solution. Generally, the aqueous phase contains the biological analyte.

The density of the organic phase is preferably higher than that of the aqueous phase that contains the biological analyte. Examples of such organic phases include haloalkanes (e.g. dichloroethane and dichloromethane), aromatics (e.g. benzyl alcohol), but more generally, any solvent which has a low solubility with the aqueous phase but a higher density than the aqueous phase can be used.

The phases can be provided in a 1:1 preferably a 2:1 or 5:1 or 10:1 vol:vol (aqueous:organic) ratio. Where the aqueous phase is provided in a greater volume than the organic phase, the ratio of aqueous to organic phase can additionally be 10:1, 50:1 or 100:1. Alternatively, the volume of the organic phase can be minimised compared with the volume of the aqueous phase.

The amount of the phases used will depend on the size of the centrifuge tube used. The aqueous phase can therefore be provided in an amount of 10 μL to 1 L, for example 100 μl to 100 ml, preferably 1 ml to 10 ml, more preferably in a volume of 1, 2, 3, 4, 5, 6, 7, 8, 9, 10 ml.

The amount of the organic phase can be determined using the ratios discussed above. Generally the volume of the organic phase is in the region of 5 μl to 1 L, for example 10 μl to 100 ml, preferably 100 μl to 10 ml, more preferably 1 ml to 5 ml.

In a particularly preferred feature of the first aspect, the method is carried out in an eppendorf tube with a capacity of 1 to 2 ml. The volume of aqueous phase used is therefore in the region of 0.5 ml to 1.9 ml, preferably 1 to 1.5 ml. It will be appreciated that it is preferential to maximize the volume of the aqueous phase in order to have more analyte/nanoparticles available.

It will be appreciated that the resulting droplet has a very high biological analyte content and a minute volume, while the concentration of other materials (such as impurities, background analyte, etc) in this droplet will be comparable to those in the starting bulk aqueous phase. As discussed above, the droplets can be combined to increase the biological analyte content even further, while keeping the concentration of impurities approximately consistent. The percentage of nanoparticles collected in these droplets from the initial bulk concentration is dependent on the interfacial area and the total number of nanoparticles, and is preferably 90% or above, more preferably 99% or more. The droplets can be diluted with ultra-pure water allowing the levels of other materials to be reduced several orders of magnitude in a single purification step. For example, if the volume of the drop is 0.5 μl from an initial volume of 1.5 ml then a single step will reduce the concentration of other materials by approximately 3000 times.

As set out above, the mixture is centrifuged until a sedimented aqueous droplet comprising the nanoparticle bound biological analyte is formed. The speed and time of centrifugation will depend on the particles and the bound biological analyte. It will be appreciated that the highest possible speed of centrifugation (RCF) should be selected for the particular sample, avoiding the occurrence of aggregation and denaturation of biological samples. The method can be carried out at speeds of up to ≈1,000,000 g with speeds of 1-50,000 g such as 1,000 g, 2,000 g, 5,000 g, 10,000 g, 20,000 g, 30,000 g, 40,000 g or 50,000 g being preferred. Preferentially, the time of centrifugation is 0.5-120 minutes. More preferably 1 minute, 5 minutes, 10 minutes, 15 minutes, 20 minutes, 30 minutes, 45 minutes, 60 minutes, 90 minutes or 120 minutes.

The skilled person will understand that the exact time of centrifugation and speed of centrifugation are not crucial to obtain purification of the biological analyte, provided that the conditions are sufficient to achieve equilibrium and aggregation/denaturing of the biological analyte are avoided. Instead, the skilled person will understand that the speed of centrifugation should by default be the highest possible on the centrifuge that is being used, while the centrifugation time should be minimized (e.g. around 5 minutes or less). If aggregation or denaturing is observed, then the RCF should be reduced, while if no droplet is being formed then either the time should be increased or the interfacial area should be reduced or the number of available nanoparticles should be increased.

It has been noted that it is possible to separate different size nanoparticles using the centrifugation method. This allows the isolation and purification of two different biological analytes using two populations of nanoparticles, wherein the nanoparticle populations are of two different sizes.

The first aspect of the present invention therefore further provides a method of purifying two separate biological analytes from a sample comprising;
incubating a sample comprising a first and second biological analyte with a first and second population of nanoparticles, wherein the first biological analyte binds to the first population of nanoparticles and the second biological analyte binds to the second population of nanoparticles;
adding an organic phase or an aqueous phase as necessary to produce a biphasic system
centrifuging the mixture until a sedimented aqueous droplet comprising the first nanoparticle bound biological analyte is formed; and
separating the resulting sedimented aqueous droplet
centrifuging the resulting supernatant until a sedimented aqueous droplet comprising the second nanoparticle bound biological analyte is formed; and
separating the resulting sedimented aqueous droplet
   wherein the organic phase is immiscible with water and has a density greater than greater than 1.0 g/cm³.

It will be appreciated that one or more droplets can be combined to provide a larger volume of ultra concentrated biological analyte. The level of concentration is limited only by the electrostatic repulsions between the nanoparticles and the physical volume that the particles occupy In particular, densities of at least 4.5 g/cm³ or above can be produced using the claimed invention.

The second aspect of the invention relates to a biological analyte purified by the method of the first aspect of the invention.

All preferred features of the first aspect of the invention also relate to the second aspect of the invention.

The invention may be put into practice in various ways and a number of specific embodiments will be described by way of example to illustrate the invention with reference to the accompanying drawings, in which:

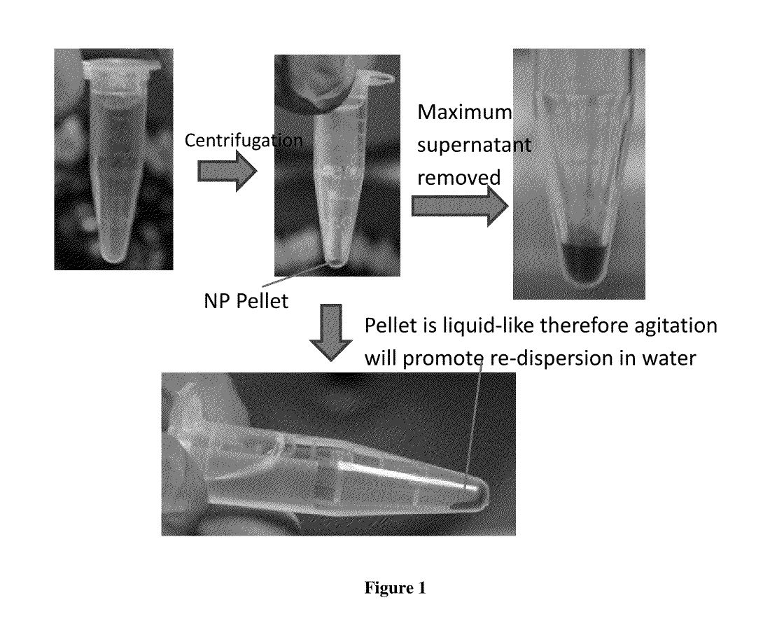

FIG. 1 illustrates the formation of a liquid-like pellet during the purification of nanoparticles using conventional centrifugation techniques;

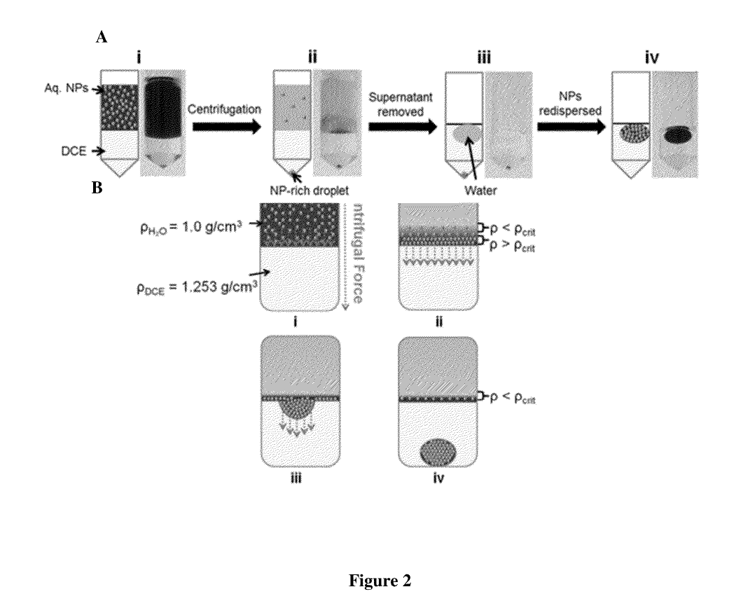

FIG. 2 illustrates a schematic and mechanism of ultra-concentration. (A) Centrifugation of an aqueous nanoparticle solution in the presence of DCE (i) leads to an extremely concentrated nanoparticle droplet being formed at the bottom of the DCE (ii). The supernatant can be easily removed and replaced with pure water (iii), if contact is made with this phase the droplet can be redispersed (iv). (B) The centrifugal force acts on the nanoparticles (i) and drives them to the interface (ii) above a critical density threshold density gradient interface is deformed (iii) and eventually a nanoparticle-rich droplet breaks off from the main aqueous phase (iv).

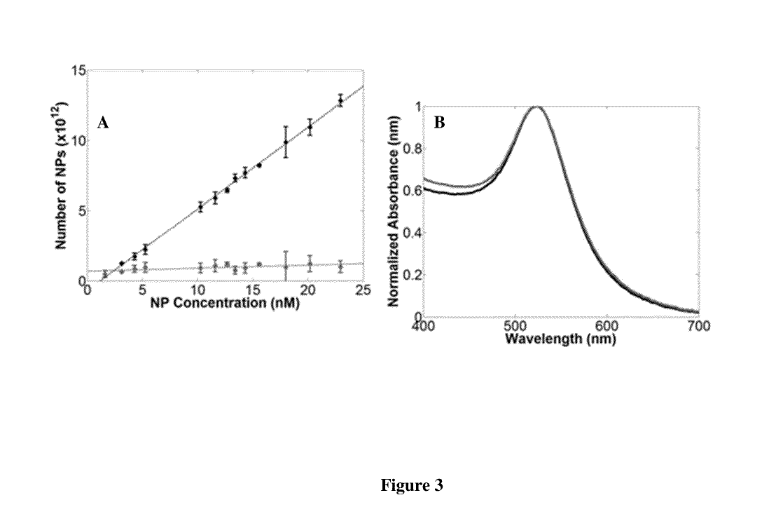

FIG. 3 provides a characterization of the initial nanoparticle concentration dependence on the nanoparticle-rich droplet formation and the stability of the ultra-concentrated nanoparticles. (A) Initial nanoparticle concentration dependence on the number of nanoparticles in the droplet (black) and residual particles in the supernatant (grey) at an initial NaCl equivalent concentration of 2.5 mM, centrifuged at 20,238 g for 15 minutes. The constant residual nanoparticles in the supernatant represents a threshold, below which no droplet is formed, while the nanoparticles in the droplet follow a y=0.58x−0.68 dependency and an R² value of 0.998. (B) Comparison of the normalized absorbance of as made nanoparticles (grey) and a 20,000 fold dilution of the nanoparticle-rich droplet (black). The fact that no red-shifted ‘shoulder’ is seen in the spectrum of the diluted sample suggests that the method does not induce aggregation.

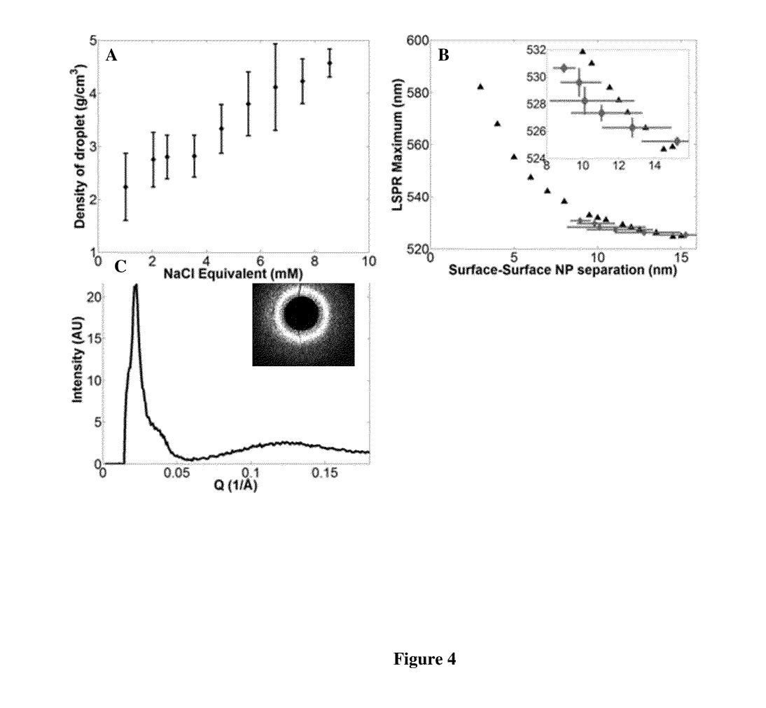

FIG. 4 illustrates the tunable properties of the nanoparticle-rich droplet as a function of salt concentration. (A) Density of the resulting nanoparticle-rich droplet as a function of the ionic strength. There is a strong dependence between the density of the resulting droplet on the NaCl concentration, due to more efficient screening of the nanoparticles charge, reaching a density of 4.57±0.26 g/cm³ at an NaCl equivalent of 8.5 mM. (B) Experimental results (grey) and simulations (black) of the LSPR maximum of the nanoparticle-rich droplet as function of the inter-particle separations agree well with each other (note: experimental separations are derived from the density). This indicates that the position of the nanoparticles with respect to each other can be tuned by the ionic strength. (C) Low angle x-ray diffraction pattern of a solution made at 4.5 mM equivalent NaCl confirms there is positional correlation between the nanoparticles, with a centre-centre separation of 29 nm;

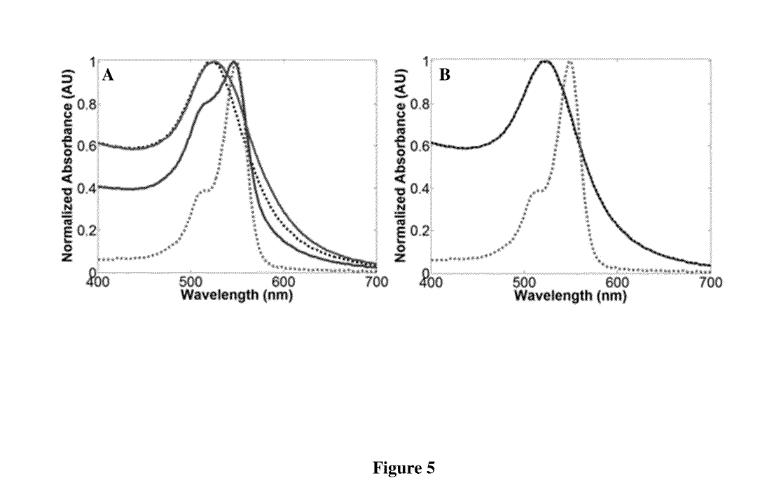

FIG. 5 provides a comparison between conventional centrifugation and this method for the purification of a dye. (A) After a single step, conventional centrifugation (b) is able to purify 95.2±0.8% of the dye Rose Bengal (c), while the second step (a) purifies an additional 90.0±3.0% of the dye to a final purity of 99.5±0.1%, when compared to as-made particles (d), a clear shift in the absorbance spectrum can still be seen. (B) After a single step forming the nanoparticle-rich droplet, and redispersing in water (a), in excess of 99.9% of the dye is removed. So much so, that there is a negligible difference in the absorbance spectrum between the as-made and purified nanoparticles. For both comparisons, the nanoparticle solutions were contaminated with 2 mM Rose Bengal and centrifugation was carried out at 20,238 g for 15 minutes;

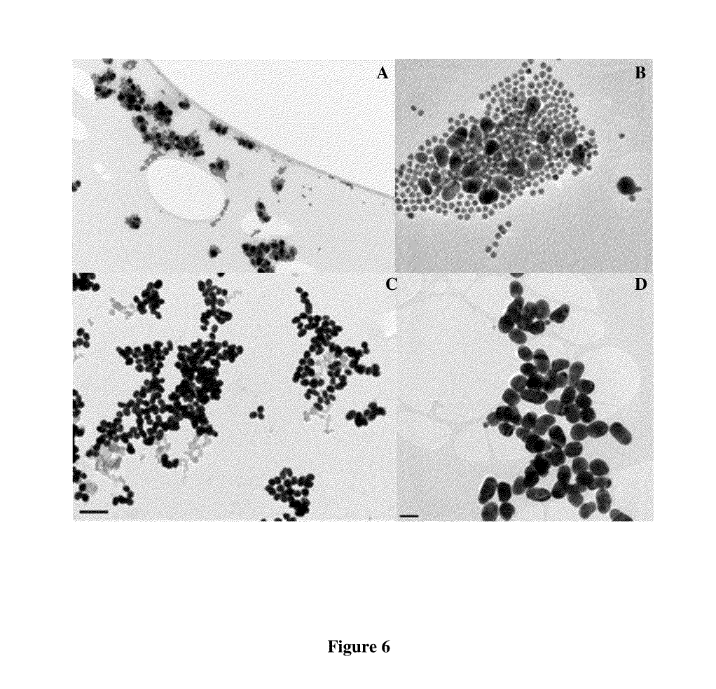

FIG. 6 provides a demonstration of size separation of NP solutions. (A) and (B), Mixture of 16 and 43 nm gold particles. (C) and (D) Separated 43 nm particles after 15 minutes at 1,503 g. Based on the analysis of over 5000 nanoparticles, there is a 99.5% removal of 16 nm nanoparticles and a 210 fold enrichment of 43 nm particles. Scale bar for (A) and (C) is 200 nm, for (B) and (D) it is 50 nm;

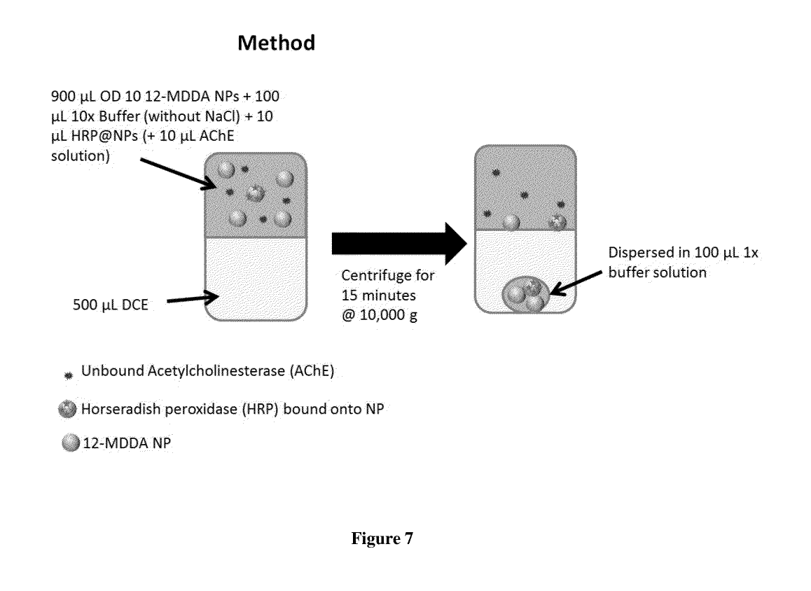

FIG. 7 illustrates an enzyme separation achieved by functionalizing nanoparticles with Horse Radish Peroxidase (HRP). Testing is carried out for enzymatic activity before centrifugation, in the droplet and in the supernatant. Enzyme activity was tested by adding a substrate that gets converted to a fluorescent product and measuring the absorbance at the maximum (where an absorbance of 4 means saturation);

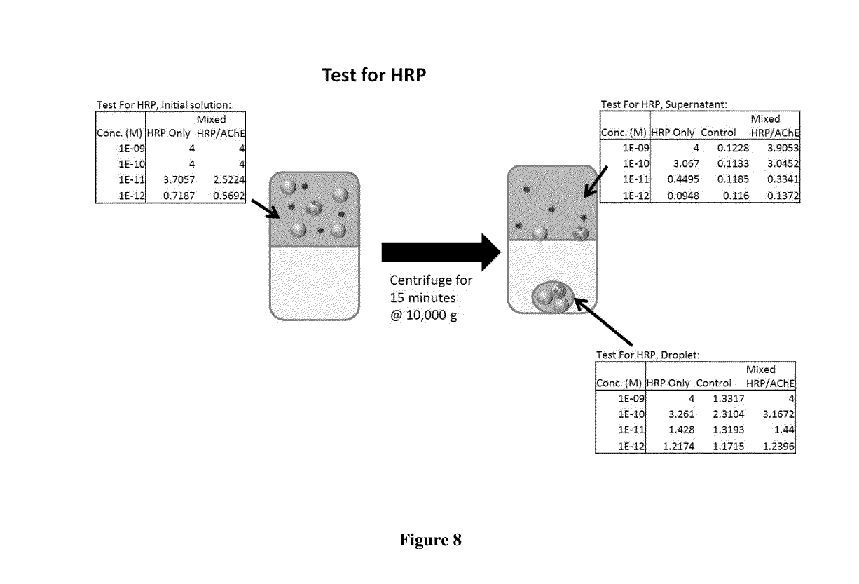

FIG. 8 illustrates tests for HRP which concluded that HRP is successfully captured by the droplet and remains active (i.e. significant denaturation does not occur). HRP can be captured even at concentrations of 1-10 picomolar;

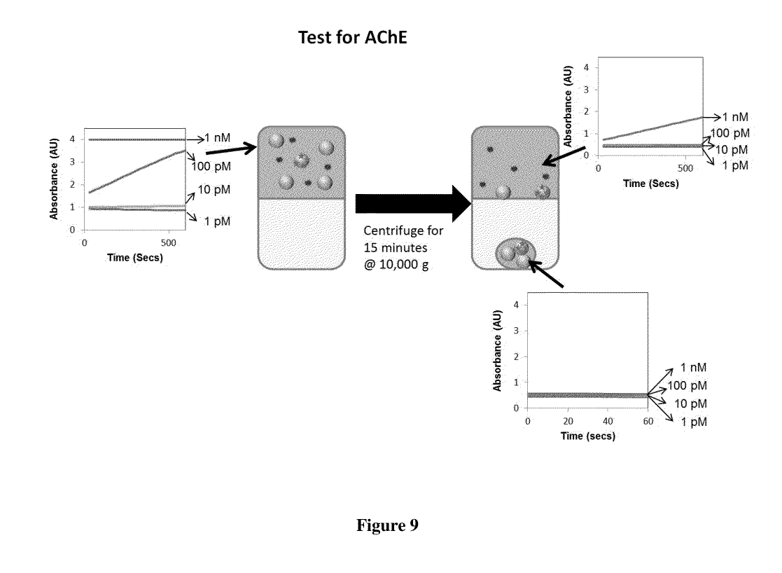

FIG. 9 illustrates tests for acetylcholine esterase (AChE) which suggest that no concentrations of AChE are seen in the purified samples. The illustrated data is a kinetic series in which a gradient in the absorbance denotes enzyme activity;

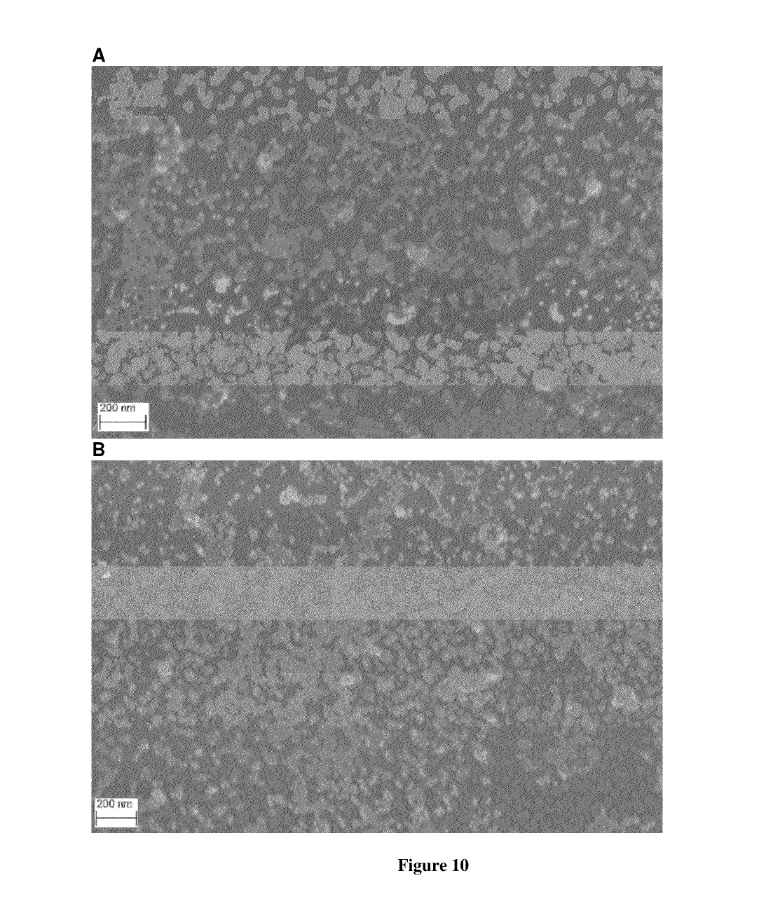

FIG. 10 illustrates SEM images demonstrating size separation. (A) and (B) Mixed sample of 16 and 43 nm nanoparticles in a 1:1 (vol/vol) ratio at as-made concentrations. (C) and (D) nanoparticles extracted from the nanoparticle-rich droplet after a 15 minute centrifugation at 1,503 g;

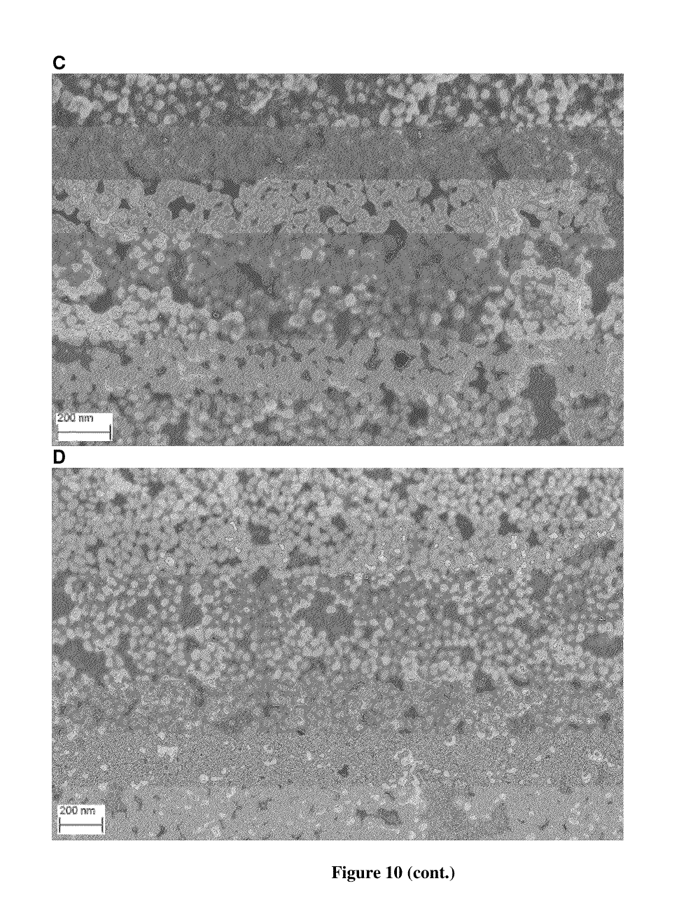

FIG. 11 shows a schematic of volume measurements of the nanoparticle-rich droplets. The droplets were sandwiched and photographed between two coverslips with a known height. From the known dimensions of the coverslips, ImageJ was used to determine the area of the circle from the top-view, followed by multiplication by the height to determine the volume.

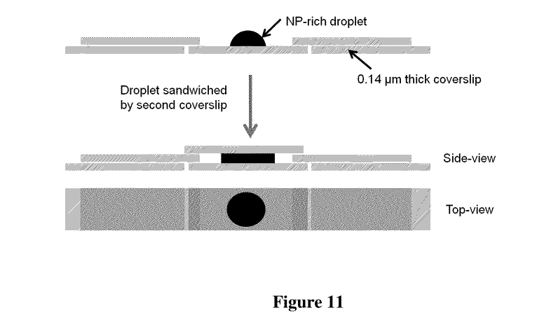

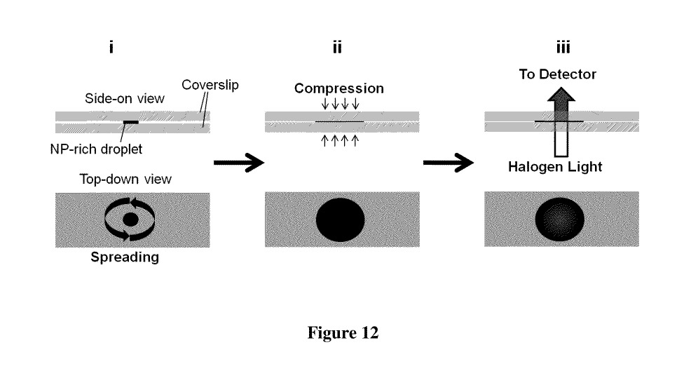

FIGS. 12 and 13 show LSPR measurements, analysis and peak broadness. FIG. 12 shows a schematic of the transmission measurements—the nanoparticle-rich droplet is first sandwiched between two coverslips, then spread to reduce the height (i), followed by compression (ii) towards the centre of the droplet. Once the droplet is sufficiently thin that a red coloration is seen, transmission measurements are then performed (iii);

FIG. 13 shows (A) the obtained absorbance spectra (black) which are fitted to a 6^{th} order polynomial (grey) and then normalized. The maximum of the polynomial is taken to be the LSPR maximum and the wavelength shift between the maximum and the red-shifted half maximum is defined as the half-width at half maximum. (B) and (C) The resultant full spectra and close up of the peaks, respectively, show a clear red shift as the NaCl concentration is increased, indicating control over the separation of the NPs. (D) The HWHM remains approximately uniform at these ionic strengths, suggesting the shift in the LSPR maximum is not due to aggregation;

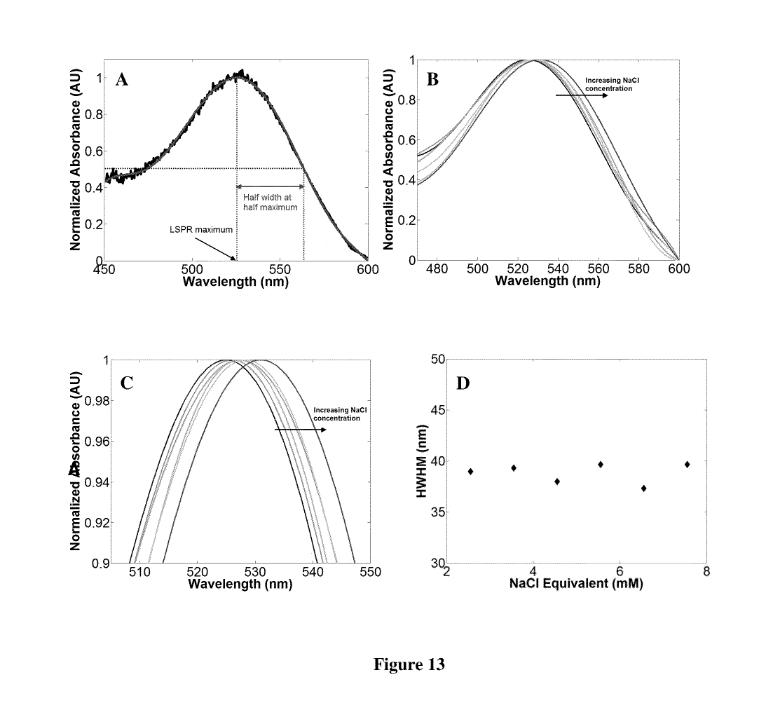

FIG. 14 shows FIT Computer Simulations. (A) Normalized absorbance for gold nanoparticles with various surface-to-surface separations. (B) The electric fields at the wavelength of the plasmonic resonance for various nanoparticle separations.

The invention will now be illustrated with reference to one or more non-limiting examples;

### EXAMPLES

Equipment used: UV-Vis measurements performed with a Nanodrop 2000c spectrometer using PMMA cuvettes. An Eppendorf 5424 centrifuge with a FA-45-24-11 rotor with a radius of 8.4 cm was used, capable of holding 24×2 ml centrifuge tubes at an angle of 45° with a maximum relative centrifugal force (RCF) of 20,238 g (14,680 RPM). A Beckmann Coulter DelsaNano C dynamic light scattering machine was used for hydrodynamic particle sizing and zeta potential measurements. A Leo Gemini 1525 FEGSEM scanning electron microscope and a JEOL 2010 transmission electron microscope were used to size the nanoparticles and demonstrate size separation. A Mettler-Toledo SevenGo Duo pro pH/ion/conductivity meter was used for conductivity measurements.

In all cases, ultra-pure water with a resistivity of 18.2 MΩ·cm was used. All chemicals were purchased from Sigma-Aldrich UK and used without further purification. Chemicals used: HAuCl₄.≈3H₂O (f.w. 339.79 (anhydrous), 99.999% trace metal basis). Trisodium citrate dihydrate (f.w. 294.10, ≧99%). 12-Mercaptododecanoic acid (MDDA, f.w. 232.38, 99%). Methanol (MeOH, CHROMASOLV, HPLC grade, ≧99.9%). 1,2-Dichloroethane (DCE) (ACS reagent, ≧99.0%)

MDDA stabilized 16 nm gold nanoparticles were prepared as described in Turek et al, ACS Nano 2012, 6, 7789-7799. Briefly: 8.6 mg HAuCl₄.3H₂O (5 mg gold) was dissolved in 95 ml H₂O and brought to 100° C. Under stirring, 5 mL 13.6 mM sodium citrate solution was then added to the refluxing mixture. The initially faint-yellow solution gradually turned dark-blue followed by wine-red over a period of 10 minutes. At this point the nanoparticles were measured to have a hydrodynamic diameter, electrophoretic mobility, and zeta potential of 20±4 nm, −3.60×10⁻⁴ cm²/Vs, and −47.0 mV respectively. The size of the particles was verified to be 16.3±2.4 nm using an SEM and TEM. Once the reaction had gone to completion (typically 15 minutes), the temperature was reduced to 60° C. This was followed by the addition of MDDA (5 mg) dissolved in MeOH (0.5 ml). The functionalization was allowed to continue for at least 1 hour to ensure complete monolayer coverage, after which the mixture was allowed to slowly cool with continuous stirring for at least an extra hour. The excess MDDA precipitated out and was removed by filtration. The functionalized nanoparticle typically had a hydrodynamic diameter of 26±7 nm, electrophoretic mobility of −4.37×10 cm²/Vs and a zeta potential of −57.0 mV. The solution typically had a conductivity of 217 μS/cm.

43 nm gold particles were synthesised as above with the exception of adding 5 mL 6.39 mM sodium citrate solution to the refluxing gold salt solution. These particles were not subsequently functionalized with MDDA. After synthesis, the concentration of the citrate ions in the solution was adjusted to be equivalent to that of the 16 nm solution. The final particles had a hydrodynamic diameter of 64.7±30.5 nm, while SEM showed an average particle size of 43±4 nm.

In all experiments a 2 mL centrifuge tube, with 0.5 mL DCE and 1 mL nanoparticle solution was used. To pellet the 16 nm particles the maximum RCF setting on the centrifuge of 20,238 g for 15 minutes was applied. In the size-separation experiments, a 1:1 (vol:vol) mixture of 16 nm and 43 nm particles was used and centrifuged at 1,503 g for 15 minutes. The pellets were extracted by pipetting out the residual aqueous phase, followed by extraction of the pellet encapsulated in a small amount of DCE which was then allowed to evaporate off.

X-ray experiments were carried out on the high brilliance beamline 122 at Diamond Light Source (DLS), UK. The synchrotron X-ray beam was tuned to a wavelength of 0.688 Å (E=18 keV), having beam dimensions of approximately 320×250 μm, and had a typical flux of 10¹² photons 1/s. The distance between the sample and detector was set at 2.2 m, and the 2-D diffraction patterns were recorded on Pilatus 2M detector. Silver behenate (a=58.38 Å) was used to calibrate the low-angle X-ray diffraction data. Diffraction images were analysed using the IDL-based AXcess software package, developed in-house by Dr A. Heron (Seddon et al, Philos. Trans. R. Soc., 2006, 364, 2635-2655) The measured X-ray spacings are accurate to ±0.1 Å.

These experiments have shown that in the presence of a denser organic phase, aqueous nanoparticles can be driven to the liquid-liquid interface (LLI), for example an aqueous |1,2-dichloroethane, interface by a centrifugal force. Provided that the difference in solvation energies and the line tension are high enough to prevent a transfer of nanoparticles across the interface, this leads to an increase in the concentration of particles directly adjacent to the interface. The concentration gradient also gives rise to a density gradient which once a critical density is reached, induces instabilities at the interface leading to the formation of nanoparticle rich droplets which sediment and merge at the bottom of the centrifuge tube (FIG. 2A). The critical density for this process is related to: the difference in density between the organic and aqueous phase; and the interfacial tension for the interface, for example, a water |1,2-dichloroethane (DCE) interface has an interfacial tension of 28.1 mN/m at 20° C.; the DCE has a density of 1.253 g/cm³ at 20° C.

The nanoparticle concentration in these droplets is high enough that electrostatic repulsion, screened by the ions of electrolyte, may induce local, short range order. The inter-particle spacing can be further controlled through the concentration of electrolyte, the increase of which will decrease the screening length. The resulting nanoparticle concentration levels at appropriate ionic strengths can exceed 3.7 g of Au per mL of solution, reaching densities of 4.5 g/cm³.

It is possible to also use low ionic strength aqueous solutions (NaCl equivalent concentration of 1.0-8.5 mM) to limit nanoparticle adsorption and facilitate a concentration gradient at the LLI. By doing so, an aqueous droplet containing an extremely high nanoparticle concentration can be formed at the bottom of the DCE layer. Experimentally this was performed in a 2 mL centrifuge tube, consisting of 0.5 mL DCE and 1 mL nanoparticle solution, with a total number of nanoparticles between 9.4×10¹¹-1.4×10¹³. To determine how many nanoparticles transfer from the bulk to the droplet, the initial nanoparticle concentration in the bulk aqueous phase was varied between 1.5 and 23 nM (FIG. 3B). A seemingly linear relationship was observed for the number of particles confined to the droplet with increasing initial concentration in the bulk of the aqueous phase; the percentage of the initial nanoparticles confined to the droplet increased from 50% at 1.5 nM to 93% at 23 nM as determined through UV-Visible spectroscopy. However, the number of residual particles in the supernatant remained approximately constant, at a value of ≈9.4×10¹¹ nanoparticles irrespective of the initial concentration.

This constant value is a threshold for the minimum number of nanoparticles needed for this method to work—below this no droplet is formed. Based on these observations, the mechanism that is proposed for the droplet generation is shown in FIG. 2B. Upon centrifugation the nanoparticles in the aqueous phase are driven to the interface (FIG. 2B i), if the initial number of particles is above the threshold value, then the local density of the aqueous side of the interface is increased (FIG. 2B ii) to the point where it is energetically more favourable for ‘extra’ interfacial area to be formed (FIG. 2B iii). Once the density gradient is established the droplets break away from the interface and sediment at the bottom of the DCE phase (FIG. 2B iv). At this point, the residual particles in the supernatant are insufficiently concentrated to obtain a sufficient density gradient for this process to be repeated.

Since the formed droplet is an extremely concentrated gold nanoparticle solution, we need to significantly dilute the sample to recover the absorbance. The droplet, formed from an aqueous solution containing an equivalent of 1 mM NaCl (as assessed from conductivity measurements), revealed the absorbance at 525 nm to be 20300±19%. Using an extinction coefficient of 4.916×10⁸ l/(Mcm)²¹ the nanoparticle concentration in the droplet was determined to be ≈41.3 μM or 2.49×10¹⁶±4.70×10¹⁵ nanoparticles/mL. Given a weight per nanoparticle of 4.40×10⁻¹⁷±1.94×10⁻¹⁷ grams, the total gold content of this solution is 1.10±0.53 grams Au/mL. Interestingly the density of this solution was determined to be 2.24±0.64 g/cm³. It should be noted that the density and dilution values are in agreement on the estimated nanoparticle concentrations—i.e. from the density (assuming a negligible density contribution from the ions and nanoparticle functionality) the weight of gold in the solution would be 1.30±0.67 g/mL.

It was decided to test whether the salt concentration can control the concentration of nanoparticles by altering the screening length. It then follows that an increase of the ionic strength leads to higher nanoparticle concentrations. This was verified by monitoring the density of the resulting solution as a function of the ionic strength of the initial pre-concentrated solution (1.0-8.5 mM equivalent NaCl). As shown in FIG. 4A, a strong effect of NaCl content on the concentration of nanoparticles in the droplet is observed. Surprisingly, at an initial NaCl concentration of 8.5 mM, the resulting nanoparticle-rich droplet has a density of 4.57±0.26 g/cm³ and is therefore higher than even a saturated Clerici solution at room temperature. This density corresponds to a solution which is composed of almost 20 vol % gold. To the best of the inventors' knowledge, this is the highest density aqueous solution that has been demonstrated at room temperature; however, it should be stressed that higher densities still are likely to be accessible with this method. Furthermore, the nanoparticle concentration of this solution is 9.09×10¹⁶ nanoparticles/mL or 3.76 grams of gold per mL the optical density at 525 nm is therefore expected to be in excess of 74,000. Taking the surface of the MDDA as the edge of the physical radius of nanoparticles (and assuming this functionality to provide a 1.5 nm steric coating), then this colloidal solution has an active surface area of 73 m²/mL.

The strong dependence of the density on the NaCl concentration suggests that the separation between the particles is dictated by the ionic screening. If metallic nanoparticles get close enough to each other, the coupling of localized plasmons in each of them leads to a red shift of the localized surface plasmon resonance (LSPR). Transmission measurements of the resulting solutions (FIG. 4B) made between 2.0-8.5 mM equivalent NaCl confirms that a red-shift between 525.2±0.3 to 530.6±0.2 nm is observed as the NaCl concentration is increased. From the densities of the solutions at these NaCl concentrations, the inter-particle separations were estimated by assuming the particles reside as far away from each other as possible and a negligible contribution from the counter-ions and functionality to the overall density of the solution. A comparison of spectra obtained from Finite Integration Techniques (FIT) computer simulations suggests that these LSPR maxima correspond to gold surface—gold surface separations between neighbouring nanoparticles of 10-15 nm. These figures are backed up further by X-ray diffraction (FIG. 4C). A nanoparticle-rich droplet made at 4.5 mM NaCl equivalent shows a sharp interference peak in the small angle region which was interpreted as the first order peak of the particle-particle structure factor. This peak indicates that the average centre-centre inter-particle separation is 29 nm (or 13 nm surface-surface separation). Working back from the density, the estimated centre-centre separation for this sample is 28.5 nm, which is in excellent agreement.

Surprisingly, despite such high concentrations, there is no evidence of aggregation. FIG. 3B shows a comparison between the normalized spectra of ‘as-made’ nanoparticles and a 20,000 fold diluted sample from a nanoparticle-rich droplet made at 1 mM NaCl. Given even mild aggregation, the broadness of the dipole peak should increase, however this is not observed in the diluted nanoparticles' absorbance spectra. The lack of aggregation is likely due to the exceptional stability of MDDA stabilized nanoparticles. Perhaps more surprising is that even citrate stabilized particles can be concentrated in this manner without initial aggregation. However it should be noted, that citrate stabilized nanoparticles have a short-lived stability and aggregate irreversibly within approximately 30 minutes of droplet formation. Despite this, it is hypothesised that the initial stability of citrate particles at these concentrations is due to the fact that the forces acting on the nanoparticles with this method are the same as during conventional centrifugation. Consequently if the nanoparticles do not aggregate during conventional centrifugation, the probability that the nanoparticles will be stable in the high density droplet is greater—however this stability may be short lived. Naturally, the versatility of this method can be extended to systems other than Au nanoparticles as long as the nanoparticles to have a sufficiently high sedimentation coefficient.

### Nanoparticle Purification and Size Separation.

Dilution of the concentrated droplet back to as-made concentrations with DI water effectively acts as a purification mechanism. The LLI confines the particles to the droplet, which together with the high density of this solution makes its separation from the supernatant trivial. To test the levels of purification that can be achieved, a dye (Rose Bengal) was added to the aqueous nanoparticle solution (with a total of 8.9×10¹² nanoparticles) at a concentration of 2 mM. Conventional centrifugation was compared to the droplet generation method, as is shown in FIGS. 5A and B. After a single step conventional centrifugation removed 95.2±0.8% of the dye, while the second step removed a further 90.0±3.0%, to give a total purity of 99.5±0.1% after 2 steps. Conversely, a single purification step with our method leads to purity levels that could not be accurately measured by UV-Visible spectroscopy (due to an almost perfect overlap between the purified and uncontaminated nanoparticles), but is in excess of 99.9%. This purification can also be applied to reducing the ionic strength of the nanoparticle solution. For example, the conductivity of the initial nanoparticle solution was 217 μS/cm whilst diluting the droplet with ultrapure water back to the as-made concentrations leads to a reduced conductivity of 1.4 μS/cm. Reducing the ionic strength aids stabilisation of the nanoparticles through the increase in Debye screening length.

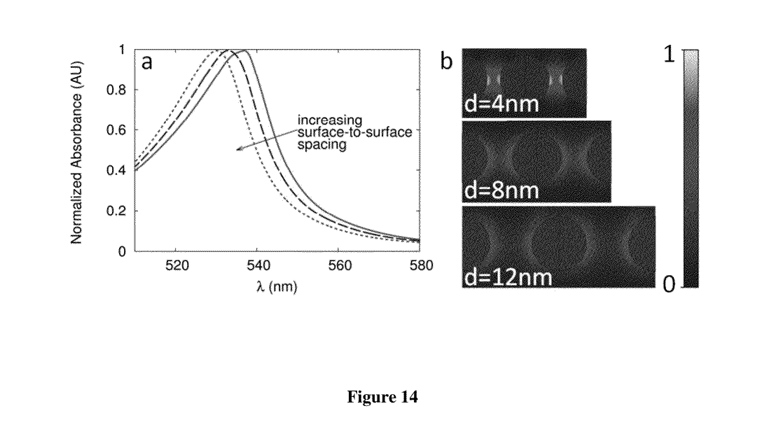

The efficiency of the purification relies on a difference in the sedimentation coefficient of the particles and dissolved species. Due to a large difference between the size of the nanoparticles and chemical species, this process proves to be extremely efficient. However the same principle can be applied to separate nanoparticles of differing sizes. For example, FIG. 16A shows a mixture of 16 nm MDDA and 43 nm citrate gold particles, mixed in a 1:1 (vol/vol) ratio of as-made solutions, this equates to approximately 6.0×10¹¹ 16 nm and 3.1×10¹⁰ 43 nm nanoparticles per mL. Centrifuging the mixture at 1,503 g for 15 minutes, results in the formation of a droplet consisting predominately of 43 nm nanoparticles, FIG. 5B. It is worth noting that optimizing the RCF may maximize the separation efficiency however this was not investigated in detail. Analysis of electron microscopy images of over 5,000 particles, showed that the ratio of 16:40 nm particles was 21.57 for the initial mixed solution, and 0.10 in the separated nanoparticle-rich droplet. This suggests a ≈210 enrichment of the 43 nm nanoparticles. This has important implications in nanoparticle synthesis where multi-modal size distributions are present. This is a common problem with more advanced nanoparticle syntheses, where despite numerous reports in the literature describing nanometer control over the dimensions of nanoparticles, secondary nano-structures also arise. As an example, the “standard” gold nanorod synthesis has exquisite control over the aspect ratio of the rods, however 3 distinct particle populations are present—rods as well as large and small spheres. Due to the properties of nanoparticles being extremely size and shape specific, these secondary particle contaminants are extremely deleterious to many of the applications that nanoparticles promise. Indeed one of the most time consuming elements of many non-spherical nanoparticle syntheses is purification and size separation of the desired product. The speed, simplicity and efficiency that this technique offers are therefore highly pertinent to researchers working with nanoparticles.

### NP-Rich Droplet Density Measurements

The mass of the NP-rich droplet was determined by extracting it from the DCE, followed by weighing it on a pre-weighed coverslip. The droplet was then sandwiched by another coverslip, with two more coverslips of a constant thickness (0.14 μm) either side acting as the height markers, FIG. 11. The area of the droplet was then calculated through ImageJ, followed by multiplication by the known height to determine the volume.

### Localized Surface Plasmon Resonance Measurements

Due to the ultra-high absorbance of the NP-rich phase, transmission measurements were performed by first sandwiching the droplet between two coverslips followed spreading and compressing the solution to yield a larger area (and thereby thinner) film. The transmitted spectrum from a halogen light source was then measured by an Ocean Optics S2000 fibre coupled spectrophotometer. The resultant spectra were then fitted to a 6^{th} order polynomial before being normalized.

### Electromagnetic Calculations for the Nanoparticle Plasmonic Resonance

Finite-integration techniques were used to solve Maxwell's equations in the frequency domain and calculate the optical spectra of gold NPs in a water solution arranged in an hcp lattice. Palik's experimental data was used to model the permittivity of gold at optical frequencies. The normalized absorbance calculated is plotted in FIG. 14A for 11-layers of gold nanoparticles with various surface-to-surface separations. It was observed that the resonance red-shifts as the separation between the nanoparticles is reduced (FIG. 13B). The spectra calculations show remarkable agreement with the experimentally obtained data plotted in FIGS. 13 (C) and (D).

The electric field distribution were calculated at the plasmonic resonance, shown in FIG. 14(B) for various nanoparticle separations. As expected, there is stronger coupling between neighbouring nanoparticles for the more dense nanoparticle solutions.

The ability to separate different size populations of nanoparticles, allows the separation of two or more biological analytes. Thus by utilising two populations of nanoparticles, each with a different size profile, and each with a different specificity ligand, it is possible to bind and therefore isolate two different biological analytes from a sample and then to separate the biological analytes by separating the two populations of nanoparticles as discussed above.

### Purification of Horse Radish Peroxidase

1 ml (OD 10) nanoparticle solution containing Horse Radish Peroxidase (HRP) bound onto some nanoparticles (HRP concentrations between 1 nM to 1 μM) was centrifuged in the presence of 0.5 ml DCE. Tests for HRP in the droplet formed from these solutions suggest that even at 1 μM HRP concentrations, the enzyme is present in the droplet. It is particularly noteworthy that the droplet needed to be diluted by ≈300 times to perform the tests, which means that the actual concentration limits for enzymatic separation may be at least an order of magnitude lower.

1. A method of purifying a biological analyte from a sample comprising;
incubating a sample comprising a biological analyte with nanoparticles;
adding an organic phase or an aqueous phase as necessary to produce a biphasic mixture;
centrifuging the mixture until a sedimented aqueous droplet comprising the nanoparticle bound biological analyte is formed;
wherein the organic phase is immiscible with water and has a density greater than greater than 1.0 g/cm³.

2. The method of claim 1 wherein the sedimented aqueous droplet is removed from the mixture.

3. The method of claim 2 wherein the sedimented aqueous droplet is subsequently diluted.

4. The method of claim 2 wherein the sedimented aqueous droplet is subsequently combined with other sedimented aqueous droplets to increase the nanoparticle content.

5. The method of claim 1, wherein the biological analyte is one or more of an enzyme, a protein, a nucleic acid, an organelle, a cell, a bacteria, or a virus.

6. The method of claim 1, wherein the nanoparticle is selected from the group consisting of metal oxide types, gold colloids, silver colloids, silica colloids, and quantum dots.

7. A biological analyte purified by the method of claim 1.

8. The method of claim 5, wherein the nucleic acid is DNA.

9. The method of claim 8, wherein the DNA is a cDNA or a plasmid.

10. The method of claim 5, wherein the nucleic acid is RNA.

11. The method of claim 10, wherein the RNA is an mRNA.
